(19) United States
(12) Patent Application Publication
(10) Pub. No.: US 2024/0216140 A1
(43) Pub. Date: Jul. 4, 2024

(54) A DEVICE FOR INSERTING A GUIDE WIRE INTO BLOOD VESSEL

(71) Applicant: VIVHEART S.R.L., Milano (MI) (IT)

(72) Inventor: Matteo MONTORFANO, Milano (MI) (IT)

(73) Assignee: VIVHEART S.R.L., Milano (MI) (IT)

(21) Appl. No.: 18/557,416

(22) PCT Filed: Sep. 15, 2022
(86) PCT No.: PCT/IB2022/058699

§ 371 Date: Oct. 26, 2023

(30) Foreign Application Priority Data
Sep. 20, 2021 (IT) 102021000024047

(51) Int. Cl. A61F 2/24; A61M 25/01; A61M 25/04; A61M 25/06; A61M 25/09

(52) U.S. Cl. CPC A61F 2/2436; A61M 25/0136; A61M 25/04; A61M 2025/0681; A61M 25/09

(57) ABSTRACT

A device for inserting a guide wire suitable for heart valve prostheses into a blood vessel is provided. The device counteracts displacement of the threadlike elements if the distal portion, to which the threadlike elements are connected, is fixed to the internal catheter which has a handle portion that allows the surgeon to move distal portion and the threadlike elements around the longitudinal axis of the catheter. The threadlike elements can be made by means of a first and a second plurality of notches offset from each other along the longitudinal axis, made on a median portion of the external catheter in order to quickly and firmly position the threadlike elements against irregular walls of a blood vessel.

### TECHNICAL FIELD

The present disclosure relates in general to catheters and more in particular to a device for inserting a guidewire into a blood vessel, wherein the device is self-centering and may be inserted into an artery for directing a guidewire through a heart valve.

### BACKGROUND OF THE INVENTION

Percutaneous valve replacement (Transcatheter Aortic Valve Replacement or more briefly TAVR) is a technique performed by inserting a catheter containing a guidewire, which is used as a guide for the implantation of a cardiac valve prosthesis. The catheter is threaded through the femoral artery and pushed up to the proximity of the defective heart valve or through the chest, for exiting the guidewire contained in the catheter near the cardiac valve so that it passes throughout it. Once the guidewire has passed throughout the heart valve, it is used to guide a valve prosthesis to the heart, where it must be positioned.

A difficulty related to this type of technique consists in the fact that the operator must be particularly skilled in order to quickly insert the guidewire through the faulty valve.

The imaging equipment currently used in operating rooms only allow a two-dimensional view and not a three-dimensional view of the guidewire approaching the defective heart valve. As a consequence, from the two-dimensional image the operator may have the wrong impression of correctly directing the guidewire to the center of the heart valve, which is also narrow and degenerated, crooked, when instead the apical end of the guidewire is going to abut against the walls of the aorta.

In any case, even if a second apparatus for images arranged so as to provide a different view were available, there would be the problem of positioning and properly orienting a catheter while pushing forward the guidewire. Moreover, the guidewire is made of yielding material so as not to damage the tissues with which it comes into contact, so it bends easily even when it should remain straight to pass through the defective valve.

This problem is even more pronounced in the so-called Valve-In-Valve technique (or more briefly VIV), in which a defective valve prosthesis is not completely removed, but is used as a frame in which a new percutaneous heart valve prosthesis is anchored. This new technique prevents the patient from the trauma of removing the old prosthetic valve, which remains attached to the heart tissue while the new valve prosthesis is attached to the old. Nevertheless this is critical because the guidewire, if pushed against the damaged portions of the old heart valve prosthesis, may cause the detachment of a part of it.

The document US2014/0207179 discloses a device, shown in FIG. 1, for centering a catheter 190 in which to pass a surgical guide wire 192. This anterior device has a plurality of thread-like elements 186 of equal length having ends fixed to a distal element 188 and to a proximal element 194. The thread-like elements 186, having thermal memory, bend and move radially away over the internal catheter 190 the distal element 188 retracting it. If the device is inserted in a substantially cylindrical portion of a blood vessel, the thread-like elements 186 abut against the blood vessel wall in a practically symmetrical manner, so that the internal catheter 190 is substantially placed at the center of the blood vessel.

The document WO2019138334 discloses a device, shown in FIG. 2a and in the detail view of FIG. 2b, comprising three catheters 1, 2, 3, with different characteristics, one inserted inside the other, to direct a guiding wire through a blood vessel and/or heart valve. Like the device of FIG. 1, it has thread-like elements 5 of equal length having ends fixed to a distal element 4 and to a proximal element 2. The distal element slides over a tubular portion 6 of the intermediate catheter 2. A more internal catheter 3, intended to conduct a surgical guide wire, tends to spontaneously bend in a more accentuated manner the more it protrudes from the tubular portion 6 of the intermediate catheter 2. An outermost catheter 1 is arranged so that the intermediate catheter 2 and the more internal 3 can be contained therein, so that the thread-like elements 5 can expand freely, exiting the outermost catheter 1 in proximity to the point where a surgical intervention is to be performed.

A drawback of this type of device is that, when inserted into a blood vessel and opened, the thread-like elements 186, 5 tend to flex along the circumferential direction as soon as they touch the blood vessel walls and move along the circumferential direction. This unexpected effect occurs in particular in large blood vessels such as the aorta. Consequently, often the catheter 190, 6, over which the distal element 188, 4 slides, is not immediately positioned in the desired way, for example in correspondence with the heart valves, so that the surgeon can only retract the thread-like elements 186, 5 into the external catheter 180, 1, and let them come out again until they are positioned in the desired way.

### SUMMARY

An object of the present disclosure is to provide a device for inserting a guide wire suitable for heart valve prostheses into a blood vessel, defined in the attached claim 1, which solves at least partially the aforementioned drawbacks.

Tests carried out by the applicant with different prototypes have shown that it is easier to counteract unwanted displacements of the thread-like elements if the distal portion, to which the thread-like elements are connected, is fixed—for example by welding or gluing or other valid joining method—to the internal catheter and the internal catheter has a handle portion which allows the surgeon, by pulling it, to modulate the folding of the thread-like elements, around the longitudinal axis of the catheter. Furthermore, thanks to the fact that it is possible to shorten the distal portion of the external catheter with respect to the longitudinal axis, by withdrawing the internal catheter, it has been noted that it is particularly convenient to make the thread-like elements by means of a first plurality of notches and a second plurality of staggered notches between them along the longitudinal direction, carried out on a median portion of the external catheter, in order to quickly and firmly position the thread-like elements against irregular walls of a blood vessel.

Preferred embodiments are defined in the dependent claims.

### BRIEF DESCRIPTION OF THE DRAWINGS

FIG. 1 shows a prior device for positioning a catheter carrying a surgical guide wire.

FIG. 2a is an overall view of another prior device for positioning a catheter carrying a surgical guide wire.

FIG. 2b is a detail view of the prior device of FIG. 2a.

FIG. 3 shows profile, top and detail views of an exemplary embodiment of this disclosure of a guide wire insertion device suitable for valve prostheses, in an extended configuration.

FIG. 4 shows profile, top and detail views of the device of FIG. 3 in an expanded configuration.

FIGS. 5a and 5b show in sequence how the thread-like elements fold/unfold by pulling/pushing a handle portion of the internal catheter of the device of FIGS. 3 and 4.

FIG. 6 is a detail view of the thread-like elements of the device of FIGS. 3 and 4 folded radially outwards.

FIGS. 7a-7d show a triangular reinforcement portion of the thread-like elements of FIG. 6.

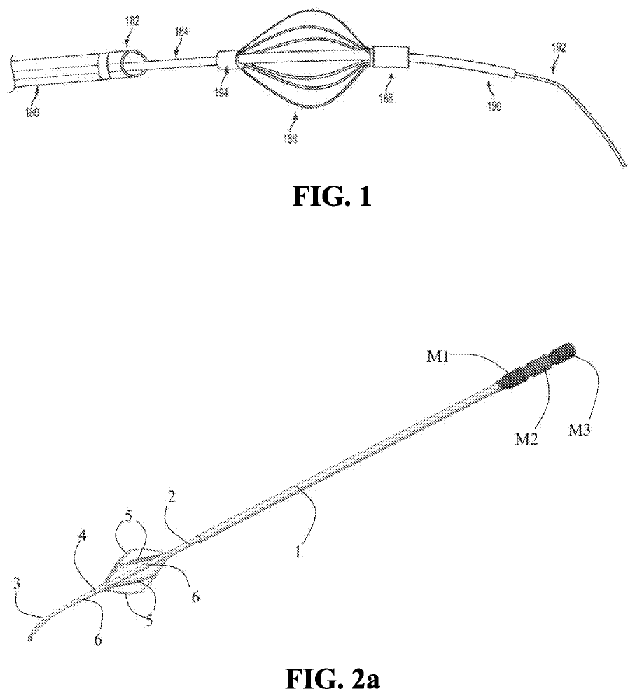

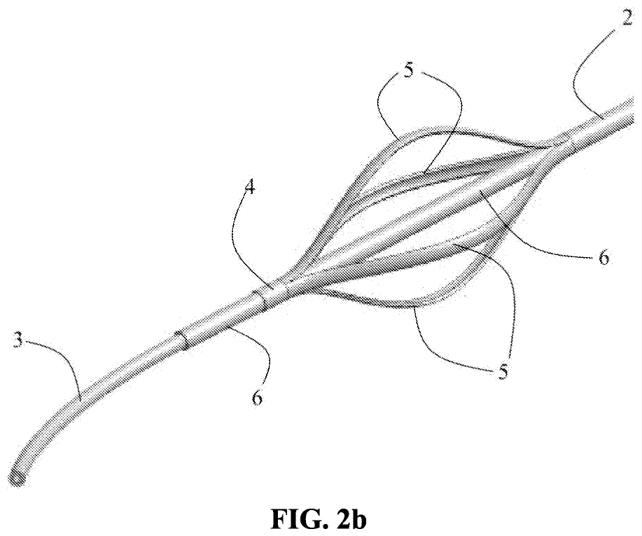

### DETAILED DESCRIPTION OF EXEMPLARY EMBODIMENTS

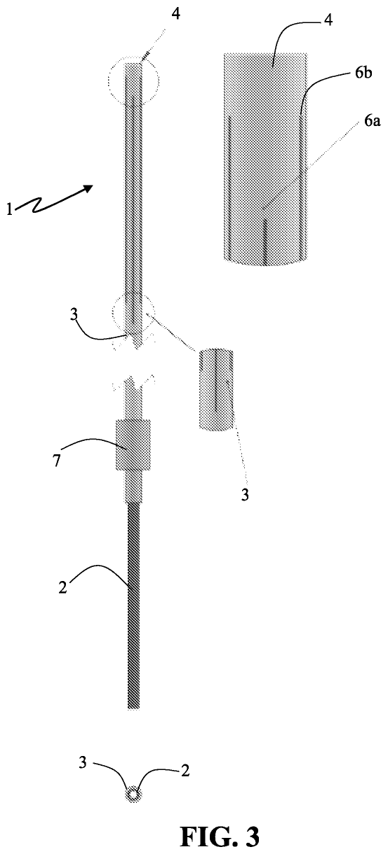

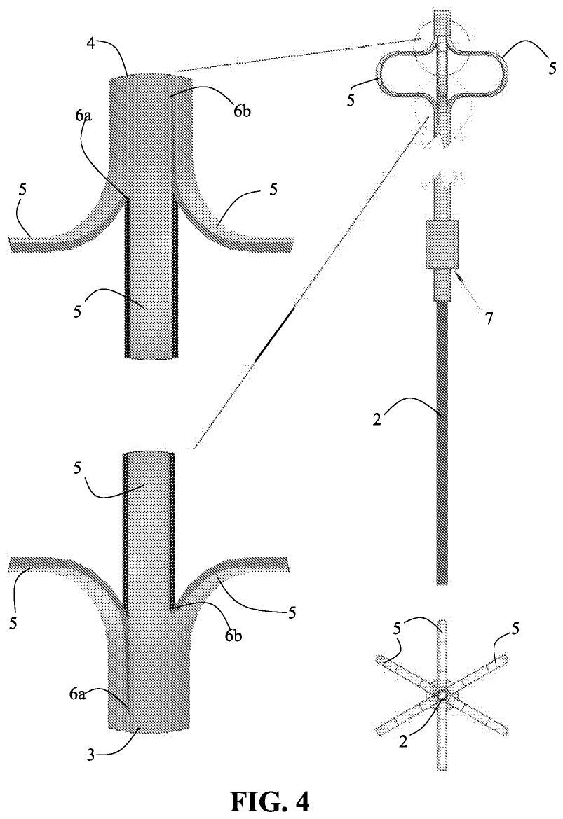

Characteristic features of the device of the present disclosure will be illustrated for simplicity with reference to the embodiment illustrated in FIGS. 3 and 4, but the skilled technician will recognize that the same observations hold, mutatis mutandis, for all the embodiments defined by the attached claims.

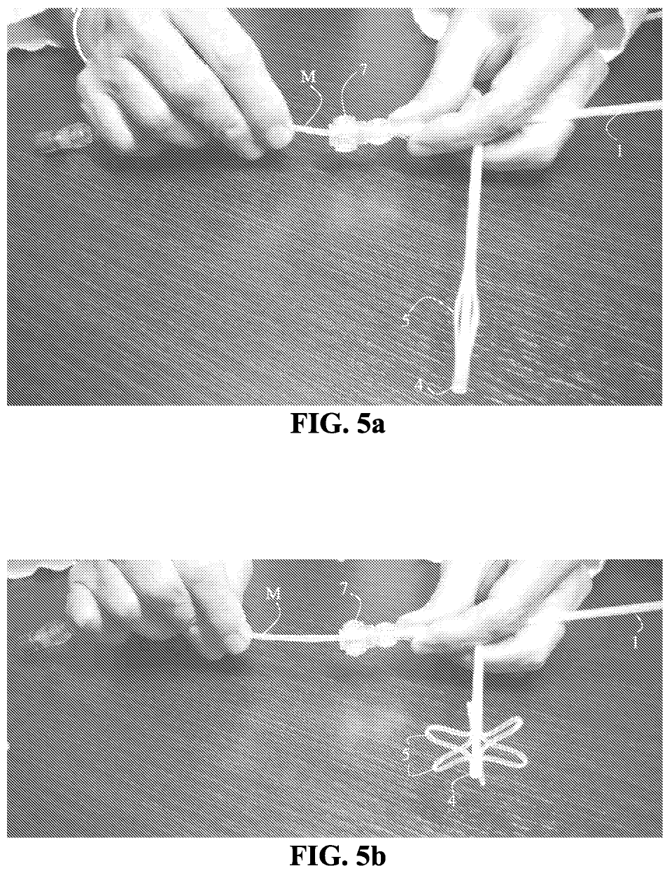

Generally, a device according to the present disclosure for inserting a guidewire suitable, for example, for heart valve prostheses in a blood vessel, comprising an external catheter 1 having a wall defining a longitudinal axis and a circumferential direction, and an internal catheter 2 inserted into the external catheter 1, having a handle portion M, shown in FIGS. 5a and 5b, configured to allow a user to advance or retract the internal catheter 2 along the longitudinal axis. The external catheter 1 defines an anchoring element to the internal walls of a blood vessel and comprises:
a distal annular portion 4 of the wall of the external catheter 1, fixed to the internal catheter 2 so as to move integrally with the internal catheter 2 when it is advanced or retracted,
a proximal annular portion 3 of the wall of the external catheter 1, sliding on the internal catheter 2, wherein the wall of the external catheter 1 does not have continuity solutions in correspondence with the distal annular portion 4 and with the proximal annular portion 3,
a median annular portion of the wall of the external catheter 1, sliding on the internal catheter 2, comprising at least a first plurality of notches directed according to the longitudinal axis and having a first length, a second plurality of direct notches according to the longitudinal axis and having a second length, in which all the notches divide the wall of the median annular portion of the external catheter 1 and define between them a plurality of threadlike elements 5 suspended above the internal catheter 2.

The threadlike elements 5 allow a self-centering/spacing of the device to the internal walls of a blood vessel because they are configured to:
folding away radially with respect to the internal catheter 2 when the handle portion M is pulled, causing the internal catheter 2 to retract along the longitudinal axis with respect to the external catheter 1, as shown in FIG. 5b, abutting against the internal walls of the blood vessel in opposite points, counteracting displacements of the internal catheter 2 transversely to the longitudinal axis,
extending along the longitudinal axis when the handle portion M is pushed by advancing the internal catheter 2 along the longitudinal axis with respect to the external catheter 1.

A characteristic of the device of the present disclosure is therefore the fact that it allows the thread-like elements 5 to be folded/stretched through the internal catheter 2 with respect to the external catheter 1 by dragging with it the distal annular portion 4 of the external catheter 1, allowing them to abut/space themselves as desired against the inner walls of the blood vessel.

In the device of the present disclosure the notches of the first plurality of notches have respective ends 6a misaligned along the circumferential direction to respective ends 6b of the notches of the second plurality of notches, so that the notches of the first plurality of notches are offset along the circumferential direction with respect to the notches of the second plurality of notches.

This feature, combined with the possibility of gradually flexing the thread-like elements 5 (due to the fact that the distal annular portion 4 is fixed to the internal catheter 2), allows to realize devices for inserting a surgical guide wire which guarantees a good positioning of the internal catheter 2 even if the blood vessel has an irregular shape, and also other functions not allowed in prior devices.

According to one aspect, the notches of the first plurality have the same length as the notches of the second plurality.

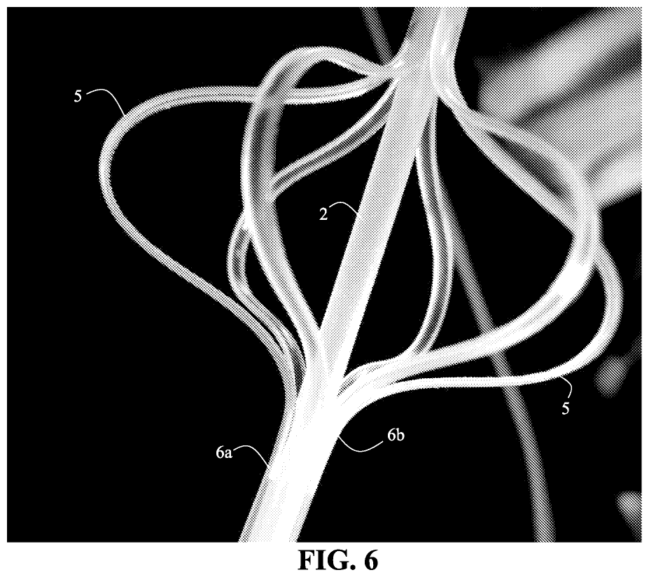

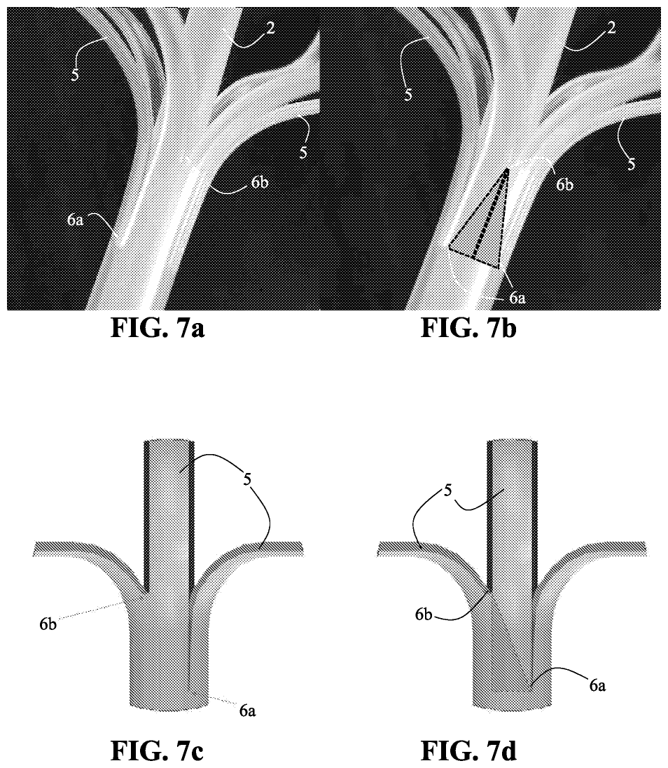

According to an optional aspect, to give greater resistance to the thread-like elements 5 to resist unwanted displacements in the circumferential direction, the notches of the first plurality of notches are orderly alternated with the notches of the second plurality of notches along the circumferential direction of the median portion of the wall of the external catheter 1, as shown in figures from 3 to 7d so that each threadlike element of the threadlike elements 5 is defined by a notch of the first plurality of notches and by a notch of the second plurality of notches. FIGS. 6, 7a and 7c show the thread-like elements 5 radially bent outwards and the triangular reinforcement portions, highlighted in FIGS. 7b and 7d, which counteract the unwanted twisting of the thread-like elements 5 when they rest against the irregular internal walls of a blood vessel.

According to an optional aspect, as shown in FIGS. 3 and 4, the notches of the first plurality of notches have their respective ends 6a aligned with each other along the circumferential direction of the median portion of the wall of the external catheter 1, and the notches of the second plurality of notches have their respective ends 6b aligned with each other along the circumferential direction of the median portion of the wall of the external catheter 1, so that all the thread-like elements 5 have the same triangular reinforcing portions.

According to one optional aspect, as shown in FIGS. 3 and 4, the device may be equipped with a hemostatic valve 7 fixed to the external catheter 1, configured so that the internal catheter 2 slides tightly therethrough along the longitudinal axis.

According to one aspect, the device of the present disclosure may comprise also a guiding catheter (not shown), preferably preformed, slidably inserted in said internal catheter 2, so as to further direct the guide wire into a blood vessel in proximity of a heart valve.

According to one aspect, the threadlike elements 5 are made of a biocompatible material suitable to be put into contact with human tissues.

According to an aspect of the present disclosure, the thread-like elements 5 can very simply be made by making the first plurality of notches and the second plurality of notches on a common catheter for surgical operations. For example, these notches can be made by machine using suitable blades, or they can be defined by laser cutting.

According to an aspect of the present disclosure, the distal portion 4, to which the thread-like elements 5 are connected, can be fixed to the internal catheter 2 by welding or gluing or other valid method of mechanical joining.

The present invention has been described so far with reference to preferred embodiments. It is understood that there could be other embodiments which refer to the same inventive concept defined by the scope of the following claims.

1. A device for inserting a guide wire suitable for heart valve prostheses into a blood vessel, comprising:
an external catheter having a wall defining a longitudinal axis and a circumferential direction,
an internal catheter inserted into said external catheter, having a handle portion configured to allow a user to advance or retract said internal catheter along said longitudinal axis,
wherein said external catheter defines an anchoring element to the internal walls of a blood vessel,
said anchoring element comprising:
a distal annular portion of said wall of the external catheter, fixed to said internal catheter so as to move integrally with said internal catheter when it is advanced or retracted,
a proximal annular portion of said wall of the external catheter, sliding on said internal catheter, wherein the wall of the external catheter does not have continuity solutions in correspondence with said distal annular portion and with said proximal annular portion,
a median annular portion of said wall of the external catheter, sliding on said internal catheter, comprising at least a first plurality of notches directed according to said longitudinal axis and having a first length, a second plurality of direct notches according to said longitudinal axis and having a second length, in which all said notches divide the wall of the median annular portion of the external catheter and define between them a plurality of threadlike elements suspended above said internal catheter;
wherein said threadlike elements are configured for:
folding away radially with respect to said internal catheter when said handle portion is pulled, causing the internal catheter to retract along said longitudinal axis with respect to the external catheter, abutting against said internal walls of the blood vessel in opposite points, counteracting displacements of the internal catheter transversely to said longitudinal axis,
extending along the longitudinal axis when said handle portion is pushed by advancing the internal catheter along said longitudinal axis with respect to the external catheter;
wherein said notches of the first plurality of notches have respective ends misaligned along said circumferential direction to respective ends of the notches of the second plurality of notches, so that the notches of the first plurality of notches are offset along the circumferential direction with respect to the notches of the second plurality of notches.

2. The device according to claim 1, wherein said second length is equal to said first length.

3. The device according to claim 1, wherein the notches of said first plurality of notches are orderly alternated with the notches of said second plurality of notches along said circumferential direction of the median portion of the wall of the external catheter, so that each threadlike element of said threadlike elements is defined by a notch of the first plurality of notches and by a notch of the second plurality of notches.

4. The device according to claim 1, in which the notches of said first plurality of notches have their respective ends aligned with each other along said circumferential direction of the median portion of the wall of the external catheter, and the notches of said second plurality of notches have their respective ends aligned with each other along said circumferential direction of the median portion of the wall of the external catheter.

5. The device according to claim 1, comprising a guide wire for heart valve prostheses inserted in said internal catheter.

6. The device according to claim 1, comprising a preformed guiding catheter slidably inserted in said internal catheter.

7. The device according to claim 1, in which said threadlike elements are made of a biocompatible material.

8. The device according to claim 1, further comprising a hemostatic valve fixed to said external catheter, configured so that said internal catheter slides tightly therethrough along said longitudinal axis.
